(19) United States
(12) Patent Application Publication
(10) Pub. No.: US 2024/0074952 A1
(43) Pub. Date: Mar. 7, 2024

(54) MASCARA AND USES THEREOF

(71) Applicant: Coty Inc., New York, NY (US)

(72) Inventor: Nicholas Quinteros, Union, NJ (US)

(21) Appl. No.: 18/461,655

(22) Filed: Sep. 6, 2023

Related U.S. Application Data
(60) Provisional application No. 63/404,387, filed on Sep. 7, 2022.

(51) Int. Cl. A61K 8/06; A61K 8/49; A61K 8/86; A61K 8/891; A61K 8/92; A61Q 1/10

(52) U.S. Cl. CPC A61K 8/064; A61K 8/4973; A61K 8/86; A61K 8/891; A61K 8/922; A61Q 1/10; A61K 2800/30

(57) ABSTRACT

Various aspects disclosed relate to provides a mascara composition. The mascara composition includes a water-in-silicone emulsion. The emulsion includes an emulsifier, a continuous phase, and a discontinuous phase. The continuous phase includes a silicone component and a wax component. The discontinuous phase includes water and a preservative component.

### CROSS-REFERENCE TO RELATED APPLICATION

This application claims the benefit of priority to U.S. Provisional patent application Ser. No. 63/404,387 entitled “MASCARA AND USES THEREOF,” filed Sep. 7, 2022, the disclosure of which is incorporated herein in its entirety by reference.

### BACKGROUND

Mascara compositions have been used for many years to increase the thickness, length and overall aesthetic appearance of human eyelashes.

### SUMMARY OF THE INVENTION

Various aspects disclosed relate to provides a mascara composition. The mascara composition includes a water-in-silicone emulsion. The emulsion includes an emulsifier, a continuous phase, and a discontinuous phase. The continuous phase includes a silicone component and a wax component. The discontinuous phase includes water and a preservative component.

Various aspects are directed towards a kit. The kit includes a container. The container includes a mascara composition. The mascara composition includes a water-in-silicone emulsion. The emulsion includes an emulsifier, a continuous phase, and a discontinuous phase. The continuous phase includes a silicone component and a wax component. The discontinuous phase includes water and a preservative component.

Various aspects are directed towards a method of making a mascara composition. The mascara composition includes a water-in-silicone emulsion. The emulsion includes an emulsifier, a continuous phase, and a discontinuous phase. The continuous phase includes a silicone component and a wax component. The discontinuous phase includes water and a preservative component. The method includes combining the emulsifier, continuous phase, and discontinuous phase.

### DETAILED DESCRIPTION OF THE INVENTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

The term “substantially free of” as used herein can mean having none or having a trivial amount of, such that the amount of material present does not affect the material properties of the composition including the material, such that about 0 wt % to about 5 wt % of the composition is the material, or about 0 wt % to about 1 wt %, or about 5 wt % or less, or less than or equal to about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The instant disclosure is related to a mascara composition. The mascara composition is advantageous in that it is a substantially waterproof mascara meaning that the composition has the ability to remain on the hair shaft despite being subjected to water such as rinsing of a face, rain, or tears. That is the mascara composition can be worn for an extended period of time without being removed by exposure to moisture (e.g., precipitation, perspiration, or the like). Alternatively, a substantially waterproof mascara can be considered one that is substantially resistant to running or smudging upon exposure to water.

In general, the mascara composition includes a water-in-silicone emulsion. The emulsion includes an emulsifier, a continuous phase, and a discontinuous phase. The continuous phase is external relative to the discontinuous phase. The continuous phase includes a silicone component and a wax component. The discontinuous phase includes water and a preservative component.

The emulsifier is in a range of from about 1 wt % to about 5 wt % of the composition, about 2 wt % to about 3.5 wt % of the composition, less than, equal to, or greater than about 1 wt %, 1.5, 2, 2.5, 3, 3.5, 4, 4.5, or about 5 wt % of the composition. Emulsifiers typically employed in the compositions of the present invention include anionic, nonionic and cationic emulsifiers. See, e.g., Encyclopedia of Chemical Technology, KIRK-OTHMER, volume 22, pp. 333-432, 3rd edition, 1979, Wiley, for the definition of the properties and (emulsifying) functions of the emulsifiers, in particular pp. 347-377 of this publication regarding anionic and nonionic emulsifiers. Examples of emulsifiers useful in the compositions of the invention include as nonionic emulsifiers, fatty acids, fatty alcohols, polyethoxylated fatty alcohols or polyglycerolated fatty alcohols, such as polyethoxylated stearyl alcohols or cetylstearyl alcohols, esters of fatty acid and sucrose, and glucose alkyl esters, in particular polyoxyethylenated C₁-C₆ alkyl glucose fatty esters, and as anionic emulsifiers, C₁₆-C₃₀ fatty acids neutralized by amines, ammonia or the alkali metal salts thereof. Examples of cationic emulsifiers include quaternary amines, amine oxides and amines, e.g., alkyl amines, alkyl imidazolines, ethoxylated amines, quaternary compounds, and quaternized esters. Cationic emulsifiers may also provide a conditioning effect. A particularly useful blend of emulsifiers is a blend of ABIL EM 90 and sorbitan olivate.

The silicone component is substantially free of cyclopentasiloxane. For example the silicone component can include less than 1 wt %, less than 0.5 wt %, less than 0.25 wt %, less than 0.05 wt %, or 0 wt % cyclopentasiloxane. The silicone component does include one or more silicones. Typically, these one or more silicones are dimethicones. The dimethicones can be characterized in terms of their respective vicsocisties. For example, the silicone component can include a blend dimethicone having a viscosity of 1 centistoke and a dimethicone having a viscosity of 2 centistoke.

The wax component typically includes a blend of different waxes. For example, the wax component includes a blend of at least two of a hard wax, a medium wax, a soft wax, or a mixture thereof. In some aspects the blend can include all three of the hard wax, medium wax, and soft wax. The terms “hard”, “medium”, and “soft” refer to the melting point of the respective waxes. The hard wax will have the highest melting point and the soft wax will have the lowest melting point. For example, the hard wax can have a melting point in a range of from about 80° C. to about 95° C. The medium wax can have a melting point in a range of from about 62° C. to about 70° C. The soft wax can have a melting point in a range of from about 45° C. to about 60° C. In certain aspects, where all three of the hard wax, medium wax, and soft wax are present, they are present in a 1:1:1 ratio (by weight). Examples of suitable waxes can include carnauba wax, candellila wax, beeswax, and polyethylene wax. However, too much wax in the semi-permanent mascara composition can adversely affect the semi-permanent properties of the wax by reducing the wear resistance of the mascara.

The preservative can include a mixture of caprylyl glycol and caprylhdroxamic acid. In some examples, the preservative is free of phenoxyethanol. Some users may have sensitivities to phenoxyethanol, so not including it can be helpful to create a mascara product that is desirable for more users.

With respect to each other, a ratio by volume of the continuous phase and discontinuous phase can be in a range of from about 4:1 to about 1:4, about 2:1 to about 1:2, less than, equal to, or greater than about 4:1, 3:1, 2:1, 1:1, 1:2, 1:3, or about 1:4.

The components and amounts of the components can control the properties of the mascara composition. The mascara composition can have a viscosity in a range of from about 2000 centipoise to about 6000 centipoise at 25° C. at a 200 s⁻¹ shear rate, from about 2500 centipoise to about 5000 centipoise, or from about 3000 centipoise to about 4500 centipoise. A proper viscosity ensures that the mascara composition can flow at a reasonable degree to be applied, yet hold when deployed on a keratinous substrate. The mascara formulation is additionally substantially waterproof. The waterproof nature of the mascara composition can be provided by the silicone component, which is repellant to water. Importantly the mascara composition is not brittle after application. That is the composition makes it possible to obtain a film that is glossy and sparingly tacky, while at the same time also being sparingly brittle or non-brittle and sufficiently flexible. This is an advantage over similar waterproof compositions, which tend to be brittle after application. Additionally, The mascara composition is suitably volumizing following application.

The mascara formulation can further include a colorant or pigment component, which can be present in the mascara formulation in a range of from about 5 wt % to about 20 wt % of the mascara formulation, about 8 wt % to about 12 wt % of the mascara formulation, less than, equal to, or greater than about 5 wt %, 10, 15, or 20 wt % of the mascara formulation. Colorants suitable for use in the present mascara formulations include, but are not limited to, dyes, pigments, lakes, and mixture thereof. (e.g., organic or inorganic pigments and colorants approved for use in eye-area cosmetics by CTFA and/or the FDA.) Exemplary inorganic pigments include particles of iron oxides (e.g., yellow, brown, red, black), titanium dioxides, iron sulfides, ultramarines, chromium oxides (e.g., green) or other conventional pigments used in cosmetic formulations. Examples of organic pigments include D&C Black No. 2, D&C Black No. 3, FD&C Red No. 40, D&C Green No. 5, FD&C Blue No. 1, and FD&C Yellow No. 5. Examples of lake dyes include various acid dyes which are laked with aluminum, calcium or barium. Additional colorants for use herein include annatto, caramel, carmine, β-carotene, bismuth oxychloride, ferric ammonium ferrocyanide, ferric ferrocyanide, chromium hydroxides (e.g., green), guanine, mica, aluminum powder, bronze powder, copper powder, manganese violet, zinc oxide. Suitable colorants along with their chemical structure are described in, e.g., 21 C.F.R. Part 74 and in the CTFA Cosmetic Ingredient Handbook, (1988), published by the Cosmetics, Toiletry and Fragrances Association, Inc. Other colorants may also be used as they are developed and determined safe.

The mascara formulation can further include a film former component. The film former component can be in a range of from about 0.20 wt % to about 15 wt % of the mascara formulation, about 10 wt % to about 15 wt % of the mascara formulation, less than, equal to, or greater than about 0.20 wt %, 0.50, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or about 15 wt % of the mascara formulation. As an example, the film former component can include polyvinylpyrrolidone. Additional suitable film formers include rosin esters, which are derived from rosin. Rosin is a solid form of resin obtained from conifers (e.g., pine tree sap). Rosin is known to include a mixture of closely related rosin acids, especially abietic acid, characterized by three fused six-carbon rings, C═C double bonds that vary in number and location, and a single carboxylic acid group. Commercial methods for obtaining rosin are also known and include, e.g., distilling the volatile turpentine from oleoresin exuded from the wound of living pine trees to obtain gum rosin, or the chemical separation of tall oil, which is a byproduct of the wood pulp industry, to obtain tall oil rosin. The carboxylic acid group of a rosin acid can be converted to an ester by reacting the acid with an alcohol. Esterification of rosin modifies the softening point, adhesiveness, cohesiveness, and melted viscosity of the material. The alcohols typically used to make rosin esters are methanol, tri-ethylene-glycol, glycerol, and pentaerythritol. Tall oil rosin is esterified with glycerol to form tall oil glycerides, a mixture of rosin acids, and esters of glycerol. Tall oil glycerides are available from, for example, Arizona Chemical Co. Glyceryl rosinate, is the ester of a rosin acid reacted with glycerol. Pentaerythrityl rosinate, sometimes referred to as pentaerythritol rosinate, is the ester of a rosin acid reacted with pentaerythritol. Pentaerythritol rosinate is commonly used as a skin conditioning agent-emollient and viscosity increasing agent, and is nonaqueous in cosmetic formulations

Rosin includes a conjugated system of C═C double bonds, which makes it susceptible to oxidation, isomerization and other chemical reactions. A common method to improve stability is to hydrogenate the rosin, for example, by the addition of hydrogen to the conjugated C═C double bonds in the presence of a catalyst to form saturated ring structures. Hydrogenated rosin esters have specific advantages over non-hydrogenated (e.g., lighter color, improved stability, and reduced skin sensitization). The hydrogenated versions of pentaerythrityl rosinate and glyceryl rosinate (i.e., pentaerythrityl hydrogenated rosinate (“PHR”) and glyceryl hydrogenated rosinate (“GHR”)) are suitable for use herein

The mascara formulation can further include carrier to help deliver the desired mascara components (e.g., pigments and film formers) to the eyelash or eyelid. In certain embodiments, the semi-permanent mascara formulation may include a volatile carrier that quickly volatilizes from the surface of the eyelashes or eyelid, leaving the desired components behind. The volatile carrier may be present at 10% to 85%, 15% to 80%, or even 20% to 70% by weight based on the weight of the composition. Nonlimiting examples of suitable volatile carriers include volatile silicones. Using silicones can obviate the need to make a container for the mascara that is resistant to degradation from hydrocarbons.

The mascara formulation can further include a rheology modifying component. The rheology modifying component can be in a range of from about 2 wt % to about 15 wt % of the mascara formulation, about 8 wt % to about 12 wt % of the mascara formulation, less than, equal to, or greater than about 2 wt %, 3, 4, 5, 6, 7, 8, 9, 10, 11, 12, 13, 14, or about 15 wt % of the mascara formulation. Examples of suitable rheology modifying components include thickeners. Thickeners for use in the present mascara formulations can be selected from the group consisting of waxes such as carnauba wax, candellila wax, and polyethylene wax; particles such as disteardimonium hectorite, kaolin, silica, and magnesium carbonate; polymers; viscous hydrocarbons; and combinations thereof. Waxes are selected to maintain the film durability of the mascara formulation.

The compositions of the present mascara formulations may comprise a safe and effective amount of a biological, chemical, nutraceutical, or pharmaceutical active, or a combination thereof. Biological actives may include prostaglandins, antimicrobials, antibacterials, biocides, preservatives, proteins, amino acids, peptides, hormones, growth factors, enzymes (e.g., glutathione sulphydryl oxidase, transglutaminase), therapeutics, oligonucleotides, genetic materials (e.g., DNA, RNA), and combinations thereof. Chemical actives may include dyes, surfactants, sensates, hair conditioners, hair dyes, hair growth agents, hair removers, hair growth inhibitors, hair styling gels, and combinations thereof. Nutraceutical actives may include proteins, preservatives, vitamins, food-additive materials, and combinations thereof. Pharmaceutical actives may include antibiotics, drugs, hair growth agents, hair removers, hair growth inhibitors, and combinations thereof.

The mascara formulation can further include an oil-soluble or dispersible additive which may include fatty alcohols such as Guerbet alcohols based on fatty alcohols having from 6 to 30, preferably from 10 to 20 carbon atoms including lauryl alcohol, cetyl alcohol, stearyl alcohol, cetearyl alcohol, oleyl alcohol, benzoates of C₁₂-C₁₅ alcohols, acetylated lanolin alcohol, etc. Especially suitable is stearyl alcohol. The oil-soluble or dispersible additive may include fatty acids such as Linear fatty acids of C₆-C₂₄, branched C₆-C₁₃carboxylic acids, hydroxycarboxylic acids, caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachidic acid, gadoleic acid, behenic acid and erucic acid and technical-grade mixtures thereof (obtained, for example, in the pressure removal of natural fats and oils, in the reduction of aldehydes from Roelen's oxosynthesis or in the dimerization of unsaturated fatty acids). Further components that can be used are dicarboxylic acids of C₂-C₁₂, such as adipic acid, succinic acid, and maleic acid. Aromatic carboxylic acids, saturated and/or unsaturated, especially benzoic acid, can be used. Additional components that can be used as the oil soluble or dispersible additive include carboxylic acid salts; alkaline soaps of sodium, potassium and ammonium; metallic soaps of calcium or magnesium; organic basis soaps such as lauric, palmitic, stearic and oleic acid, etc., alkyl phosphates or phosphoric acid esters: acid phosphate, diethanolamine phosphate, potassium cetyl phosphate

Other useful oil-soluble or dispersible additives comprise mild surfactants, super-fatting agents, consistency regulators, additional thickeners, polymers, stabilizers, biologically active ingredients, deodorizing active ingredients, anti-dandruff agents, film formers, swelling agents, UV light-protective factors, antioxidants, preservatives, insect repellents, solubilizers, colorants, bacteria-inhibiting agents, hair conditioning agents, vitamins, and the like.

There are any number of further components that can be included in the mascara formulation such as a surfactant component, a skin conditioner component, a humectant component, or another additive component. Other additives can include chamomile and cucumber extract, jojoba oil, camella oil, aloe butter, maracuja oil, cell-u-lash, marula oil, castor oil, ceramide 2, avocado butter, or a mixture thereof.

In some examples, the mascara composition can include a number of vegan components. In some further examples, the mascara composition can be a completely vegan composition. As understood herein a “vegan composition” refers to a composition including components that are not derived from an animal product and to the extent possible not tested on an animal.

The mascara formulation can be contained in a applicator assembly. A variety of conventional applicators are known in the art (e.g., molded plastic applicators and twisted-wire brush applicators). Mascara formulations may act as a paste or semi-solid on the brush, rather than as a flowable liquid. Thus, the primary method of depositing such mascaras onto the mascara brush is by inserting the brush into a supply of mascara, typically contained in a bottle or similar container, so that the mascara is deposited on the head (i.e., bristles and core) of the brush. Excess mascara is removed from the head of the brush, especially the outermost portions of the bristles spaced away from the core, via a wiper when the brush is withdrawn from the mascara container. Ideally, a suitable amount of mascara remains at least on the core of the brush for application to the eyelashes of a user. When the brush is used as intended, the lashes of a user are moved past the bristles of the brush via the spaces between the bristles such that the lashes come into contact with the mascara-containing core and, optionally, portions of the bristles (e.g., the inner portions). Thus, it is the characteristics of the mascara (e.g., coherent strength & rigidity) in combination with the applicator and wiper that provides the primary mechanism of product control and dosing. Because conventional mascaras tend to be thick enough for the mascara to remain on the core of the brush, it is not uncommon for the spacing of the bristles to be determined primarily by the desired lash separation/clustering. But with the desire to provide lower viscosity mascaras, the spacing of the bristles becomes important for providing suitable product retention on the brush as well as the desired amount of lash separation/clustering.

A suitable mascara product according to the present disclosure includes a container (e.g., bottle or the like) for storing a supply of the present semi-permanent mascara formulation; an applicator (e.g., twisted-wire brush) for transferring mascara from the container to the eyelashes of a user; and a supply of the present semi-permanent mascara formulation disposed in the container. The applicator may include a handle which is removably and/or reattachably joined to the container (e.g., with screw threads, snap collar, or the like).

The mascara composition can be formed according many suitable methods. As a non-limiting example, the method can include combining the emulsifier, continuous phase, and discontinuous phase. The combination is then mixed for a suitable amount of time to suitably combine the components and form the mascara composition. The silicone component can be added to the first stage of the second stage. However, it was found that adding the silicone component in stagged steps, e.g., ¼ to ½ of the total amount to different stages was preferable. Other components can be added to the first stage or second stage as desired.

### EXEMPLARY ASPECTS

The following exemplary aspects are provided, the numbering of which is not to be construed as designating levels of importance:
Aspect 1 provides a mascara composition, comprising:
a water-in-silicone emulsion, comprising:
an emulsifier;
a continuous phase comprising:
   a silicone component; and
   a wax component; and
a discontinuous phase comprising:
   water; and
   a preservative component.
Aspect 2 provides the mascara composition of Aspect 1, wherein the silicone component comprises one or more silicones.
Aspect 3 provides the mascara composition of any one of Aspects 1 or 2, wherein the silicone component comprises dimethicone.
Aspect 4 provides the mascara composition of any one of Aspects 1-3, wherein the silicone component comprises a blend of 1 centistoke dimethicone and 2 centistoke dimethicone.
Aspect 5 provides the mascara composition of any one of Aspects 1-4, wherein the wax component comprises carnauba wax, candellila wax, paraffin wax, polyethylene wax, or a mixture thereof
Aspect 6 provides the mascara composition of any one of Aspects 1-5, wherein the wax component comprises a hard wax, a medium wax, a soft wax, or a mixture thereof.
Aspect 7 provides the mascara composition of Aspect 6, wherein the wax component comprises at least two of a hard wax, a medium wax, a soft wax, or a mixture thereof.
Aspect 8 provides the mascara composition of any one of Aspects 6 or 7, wherein
the hard wax has a melting point in a range of from about 80° C. to about 95° C.;
the medium wax has a melting point in a range of from about 62° C. to about 70° C.; and
the soft wax has a melting point in a range of from about 45° C. to about 60° C.
Aspect 9 provides the mascara composition of any one of Aspects 6-8, wherein
the hard wax has a melting point in a range of from about 80° C. to about 95° C.;
the medium wax has a melting point in a range of from about 62° C. to about 70° C.; and
the soft wax has a melting point in a range of from about 45° C. to about 60° C.
Aspect 10 provides the mascara composition of any one of Aspects 6-9, wherein the hard wax, medium wax, and soft wax are present in a 1:1:1 ratio.
Aspect 11 provides the mascara composition of any one of Aspects 1-10, wherein the preservative component comprises a mixture of caprylyl glycol and caprylhdroxamic acid.
Aspect 12 provides the mascara composition of any one of Aspects 1-11, wherein a ratio of the continuous phase and discontinuous phase is in a range of from 4:1 to 1:4.
Aspect 13 provides the mascara composition of any one of Aspects 1-12, wherein a ratio of the continuous phase and discontinuous phase is in a range of from 2:1 to 1:2.
Aspect 14 provides the mascara composition of any one of Aspects 1-13, wherein the emulsifier comprises a blend of is Cetyl PEG/PPG-10/1 Dimethicone and sorbitan olivate.
Aspect 15 provides the mascara composition of any one of Aspects 1-14, wherein the emulsifier is in a range of from about 1 wt % to about 5 wt % of the composition.
Aspect 16 provides the mascara composition of any one of Aspects 1-15, wherein the emulsifier is in a range of from about 2 wt % to about 3.5 wt % of the composition.
Aspect 17 provides the mascara composition of any one of Aspects 1-16, wherein a viscosity of the mascara composition is in a range of from about 2000 centipoise to about 6000 centipoise at 25° C. at a 200 s⁻¹ shear rate.
Aspect 18 provides the mascara composition of any one of Aspects 1-17, wherein a viscosity of the mascara composition is in a range of from about 2000 centipoise to about 5000 centipoise at 25° C. at a 200 s⁻¹ shear rate.
Aspect 19 provides the mascara composition of any one of Aspects 1-18, wherein the mascara composition is substantially waterproof.
Aspect 20 provides the mascara composition of any one of Aspects 1-19, wherein the composition is substantially non-brittle.
Aspect 21 provides the mascara composition of any one of Aspects 1-20, wherein the composition is substantially volumizing.
Aspect 22 provides the mascara composition of any one of Aspects 1-21, wherein the composition is substantially free of cyclopentasiloxane.
Aspect 23 provides the mascara composition of any one of Aspects 1-22, wherein the composition is substantially free of phenoxyethanol.
Aspect 24 provides the mascara composition of any one of Aspects 1-23, wherein the composition is a vegan composition.
Aspect 25 provides the mascara composition of any one of Aspects 1-24, wherein a flashpoint of the composition is in a range of from about 30° C. to about 95° C.
Aspect 26 provides the mascara composition of any one of Aspects 1-25, wherein a flashpoint of the composition is in a range of from about 35° C. to about 45° C.
Aspect 27 provides the mascara composition of any one of Aspects 1-26, further comprising a pigment.
Aspect 28 provides a kit comprising:
a container; and
the mascara composition of any one of Aspects 1-27, disposed in the container.
Aspect 29 provides a method of making the mascara composition of any one of Aspects 1-28, the method comprising
combining the emulsifier, continuous phase, and discontinuous phase.
Aspect 30 provides the method of Aspect 29, wherein the silicone component is added in at least a first stage and a second stage.
Aspect 31 provides the method of Aspect 30, wherein the first stage is in a range of from about 10% to about 60% of the total amount of silicone component.
Aspect 32 provides the method of Aspect 30 or 31, wherein the first stage is in a range of from about 20% to about 40% of the total amount of silicone component.

What is claimed is:

1. A mascara composition, comprising:
a water-in-silicone emulsion, comprising:
an emulsifier;
a continuous phase comprising:
a silicone component; and
a wax component; and
a discontinuous phase comprising:
water; and
a preservative component.

2. The mascara composition of claim 1, wherein the silicone component comprises one or more silicones.

3. The mascara composition of claim 1, wherein the silicone component comprises dimethicone.

4. The mascara composition of claim 1, wherein the silicone component comprises a blend of 1 centistoke dimethicone and 2 centistoke dimethicone.

5. The mascara composition of claim 1, wherein the wax component comprises carnauba wax, candellila wax, paraffin, polyethylene wax, or a mixture thereof.

6. The mascara composition of claim 1, wherein the wax component comprises a hard wax, a medium wax, a soft wax, or a mixture thereof.

7. The mascara composition of claim 6, wherein the wax component comprises at least two of a hard wax, a medium wax, a soft wax, or a mixture thereof.

8. The mascara composition of claim 6, wherein
the hard wax has a melting point in a range of from about 80° C. to about 95° C.;
the medium wax has a melting point in a range of from about 62° C. to about 70° C.; and
the soft wax has a melting point in a range of from about 45° C. to about 60° C.

9. The mascara composition of claim 6, wherein
the hard wax has a melting point in a range of from about 80° C. to about 95° C.;
the medium wax has a melting point in a range of from about 62° C. to about 70° C.; and
the soft wax has a melting point in a range of from about 45° C. to about 60° C.

10. The mascara composition of claim 6, wherein the hard wax, medium wax, and soft wax are present in a 1:1:1 ratio.

11. The mascara composition of claim 1, wherein the preservative component comprises a mixture of caprylyl glycol and caprylhdroxamic acid.

12. The mascara composition of claim 1, wherein a ratio of the continuous phase and discontinuous phase is in a range of from 4:1 to 1:4.

14. The mascara composition of claim 1, wherein the emulsifier comprises a blend of Cetyl PEG/PPG-10/1 Dimethicone and sorbitan olivate.

15. The mascara composition of claim 1, wherein a viscosity of the mascara composition is in a range of from about 2000 centipoise to about 6000 centipoise at 25° C. at a 200 s⁻¹ shear rate.

16. The mascara composition of claim 1, wherein a viscosity of the mascara composition is in a range of from about 2000 centipoise to about 5000 centipoise at 25° C. at a 200 s⁻¹ shear rate.

17. The mascara composition of claim 1, wherein the mascara composition is substantially waterproof.

18. The mascara composition of claim 1, wherein the composition is substantially non-brittle.

19. The mascara composition of claim 1, wherein the composition is substantially free of cyclopentasiloxane or phenoxyethanol.

20. The mascara composition of claim 1, wherein a flashpoint of the composition is in a range of from about 30° C. to about 50° C.
